(19) United States
(12) Patent Application Publication
(10) Pub. No.: US 2026/0325750 A1
(43) Pub. Date: Oct. 22, 2026

(54) CANCER THERAPY SYSTEM WITH ENHANCED THERAPEUTIC MICROSPHERE AND FLUID MIXING

(71) Applicant: Boston Scientific Scimed, Inc., Maple Grove, MN (US)

(72) Inventors: Patrick A. Haverkost, Crystal, MN (US); Joel N. Groff, Delano, MN (US); Scott McGhee, Ottawa (CA); David Raab, Roseville, MN (US); McKenzie Dudley, St. Louis Park, MN (US)

(21) Appl. No.: 19/185,990

(22) Filed: Apr. 22, 2025

Related U.S. Application Data
(60) Provisional application No. 63/638,794, filed on Apr. 25, 2024.

(51) Int. Cl. A61M 5/168

(52) U.S. Cl. CPC A61M 5/16827

(57) ABSTRACT

Embodiments herein relate to cancer therapy delivery systems with enhanced microsphere and fluid mixing. In an embodiment, a cancer therapy delivery system is included having a mixing chamber. The mixing chamber defines a hemispherical tip and defines continuously tapered sides. The delivery system also includes an inflow conduit, wherein the inflow conduit is in selective fluid communication with the mixing chamber. The delivery system also includes an outflow conduit, wherein the outflow conduit is in selective fluid communication with the mixing chamber. Other embodiments are also included herein.

This application claims the benefit of U.S. Provisional Application No. 63/638,794 filed Apr. 25, 2024, the content of which is herein incorporated by reference in its entirety.

### FIELD

Embodiments herein relate to cancer therapy delivery systems. More specifically, embodiments herein relate to a cancer therapy delivery system with enhanced microsphere and fluid mixing.

### BACKGROUND

According to the American Cancer Society, cancer accounts for nearly 25% of the deaths that occur in the United States each year. Cancerous tumors can form if one normal cell in any part of the body mutates and then begins to grow and multiply too much and too quickly. Cancerous tumors can be a result of a genetic mutation to the cellular DNA or RNA that arises during cell division, an external stimulus such as ionizing or non-ionizing radiation, exposure to a carcinogen, or a result of a hereditary gene mutation. Regardless of the etiology, many cancerous tumors are the result of unchecked rapid cellular division.

Surgery is a common first-line therapy for many cancerous tumors. However, not every tumor can be surgically removed. Chemotherapy and immunotherapy are other common therapeutic approaches but can include substantial side effects. The use of radiation represents another approach. Specifically, radiation therapy aims at damaging the DNA of cancer cells so that they lose the capability to divide and proliferate, thus leading to the cell death process for the cancerous cells.

### SUMMARY

Embodiments herein relate to a cancer therapy delivery system with enhanced microsphere and fluid mixing. In a first aspect, a cancer therapy delivery system can be included having a mixing chamber defining a hemispherical tip and continuously tapered sides. The system can include an inflow conduit, wherein the inflow conduit can be in selective fluid communication with the mixing chamber. The system can also include an outflow conduit, wherein the outflow conduit can be in selective fluid communication with the mixing chamber.

In a second aspect, in addition to one or more of the preceding or following aspects, or in the alternative to some aspects, a width of a portion of the mixing chamber defining the continuously tapered sides can be less than 30 percent of a height of the portion of the continuously tapered sides.

In a third aspect, in addition to one or more of the preceding or following aspects, or in the alternative to some aspects, the cancer therapy delivery system can further include a pump and a fluid reservoir, wherein the fluid can be withdrawn from the fluid reservoir and flow into the mixing chamber via the inflow conduit.

In a fourth aspect, in addition to one or more of the preceding or following aspects, or in the alternative to some aspects, the fluid can be a saline solution.

In a fifth aspect, in addition to one or more of the preceding or following aspects, or in the alternative to some aspects, the system can further include radioactive microspheres, wherein the radioactive microspheres can be disposed within the mixing chamber.

In a sixth aspect, in addition to one or more of the preceding or following aspects, or in the alternative to some aspects, the radioactive microspheres have a density of about 3.3 g/mL.

In a seventh aspect, in addition to one or more of the preceding or following aspects, or in the alternative to some aspects, the radioactive microspheres have an average diameter of about 20 μm to about 30 μm.

In an eighth aspect, in addition to one or more of the preceding or following aspects, or in the alternative to some aspects, the fluid that flows into the fluid reservoir causes the radioactive microspheres to form a suspension with the glass microspheres that then passes out of the mixing chamber via the outflow conduit.

In a ninth aspect, in addition to one or more of the preceding or following aspects, or in the alternative to some aspects, the fluid that flows into the fluid reservoir generates a region of shear along the walls thereof of at least 0.020 Pa when the fluid flows through the fluid reservoir at a rate of at least 5 mL/minute.

In a tenth aspect, in addition to one or more of the preceding or following aspects, or in the alternative to some aspects, the fluid that flows into the fluid reservoir generates a region of shear along the walls thereof of at least 0.020 Pa covering a region extending above the bottom of the outflow conduit when the outflow conduit can be in a deployed position when the fluid flows through the fluid reservoir at a rate of at least 5 mL/minute.

In an eleventh aspect, in addition to one or more of the preceding or following aspects, or in the alternative to some aspects, the continuously tapered sides can be at an angle of less than 20 degrees with respect to a lengthwise vertical axis of the mixing chamber.

In a twelfth aspect, in addition to one or more of the preceding or following aspects, or in the alternative to some aspects, the continuously tapered sides can be at an angle of less than 10 degrees with respect to a lengthwise vertical axis of the mixing chamber.

In a thirteenth aspect, in addition to one or more of the preceding or following aspects, or in the alternative to some aspects, a region of the mixing chamber having the continuously tapered sides can have a width to height ratio of less than 1:2.

In a fourteenth aspect, in addition to one or more of the preceding or following aspects, or in the alternative to some aspects, a region of the mixing chamber having the continuously tapered sides can have a width to height ratio of less than 1:3.

In a fifteenth aspect, in addition to one or more of the preceding or following aspects, or in the alternative to some aspects, a volume of the mixing chamber up to a top of the continuously tapered sides can be less than 1 mL.

In a sixteenth aspect, in addition to one or more of the preceding or following aspects, or in the alternative to some aspects, the mixing chamber can be defined by a glass or polymeric vial.

In a seventeenth aspect, in addition to one or more of the preceding or following aspects, or in the alternative to some aspects, the inflow conduit can be at least one of a needle or a cannula.

In an eighteenth aspect, in addition to one or more of the preceding or following aspects, or in the alternative to some aspects, the outflow conduit can be at least one of a needle or a cannula.

In a nineteenth aspect, a method of delivering a cancer therapy to a patient can be included, the method including preparing a cancer therapy system, injecting a carrier fluid into an internal volume of a mixing chamber of the cancer therapy system, wherein the mixing chamber defines a hemispherical tip and continuously tapered sides, withdrawing microspheres out of the mixing chamber in the form of a mixture or suspension with the carrier fluid and conveying the same to a delivery catheter, and delivering the therapeutic microspheres to the patient using the delivery catheter.

In a twentieth aspect, in addition to one or more of the preceding or following aspects, or in the alternative to some aspects, wherein preparing a cancer therapy system includes one or more of one or more operations including priming the system, removing bubbles, and flushing the lines.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of a cancer therapy delivery system in accordance with various embodiments herein.

FIG. 2 is a sectional view of components of a cancer therapy delivery system in accordance with various embodiments herein.

FIG. 3 is a sectional view of components of a cancer therapy delivery system in accordance with various embodiments herein.

FIG. 4 is a schematic view of components of a cancer therapy delivery system in accordance with various embodiments herein.

FIG. 5 is a sectional view of portions of a mixing chamber as taken along line 5-5 of FIG. 4 in accordance with various embodiments herein.

FIG. 6 is an enlarged view of a portion of the mixing chamber including the hemispherical tip.

FIG. 7 is a schematic view of portions of a mixing chamber herein showing shear generated in accordance with various embodiments herein.

FIG. 8 is a flowchart showing operations that can be performed in accordance with various embodiments herein.

While embodiments are susceptible to various modifications and alternative forms, specifics thereof have been shown by way of example and drawings and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

Radiation therapy aims at damaging the DNA of cancer cells so that they lose the capability to divide and proliferate, thus leading to the cell death process for the cancerous cells. Brachytherapy is a form of radiation therapy where a sealed radiation source is placed inside or next to the area requiring treatment. As one form of brachytherapy, targeted radioembolization therapy can be used to treat unresectable tumors. For example, Y-90 glass microspheres can be delivered into or adjacent to a tumor through a microcatheter placed into an artery that supplies blood to the tumor. The beta radiation emitted by the Y-90 can exert a local radiotherapeutic effect on the tumor. Other radioisotopes can also be used in some types of brachytherapy.

In some approaches including the use of microspheres or other particulates, the microspheres are delivered to the patient through a catheter or other delivery line, carried along by a flow of carrier fluid and, once in the body, the microspheres or other particulates and the carrier fluid mix with blood flow and then the resulting mixture passes onto a target site in the body. However, before passing through the catheter the carrier fluid is mixed with microspheres forming a mixture or suspension that is then conveyed along to the catheter. However, due to various factors, including a substantial difference in the density of the microspheres versus the fluid, the microspheres can settle out of the mixture or suspension relatively quickly, in part even before leaving the mixing chamber. However, embodiments of cancer therapy delivery systems herein can include a mixing chamber that more effectively mixes the fluid and the microspheres and provides for more effective and consistent delivery of the microspheres. Embodiments herein improve delivery efficiency which can enable a lower carrier fluid flow rate such as 6 mL/min, 5 mL/min, or less (while still achieving satisfactory mixing of fluid and microspheres) as well as reduce the total carrier fluid volume delivered to a tumor site by requiring fewer flushes to ensure all or nearly all microspheres have been delivered. Lower carrier fluid flow rates and reduced carrier fluid volumes can be important when treating certain types of tumors such as those in the brain (such as glioblastoma) where the capacity to handle additional fluid volumes and flow rates may be limited.

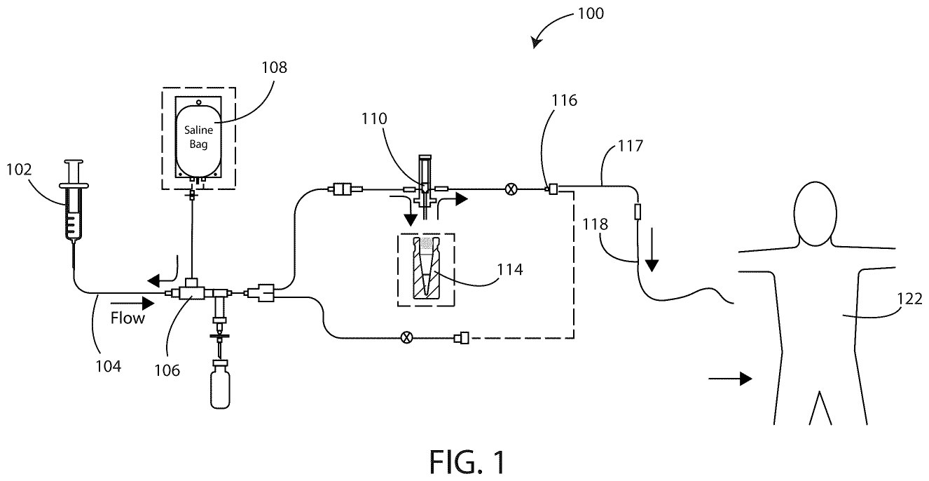

Referring now to FIG. 1, a schematic diagram is shown of components of an exemplary cancer-therapy delivery system 100 in accordance with various embodiments herein. Major parts of the cancer therapy delivery system 100 include a therapeutic fluid delivery device 102 (or pump, which in some cases can take the form of a syringe or syringe-like device), a fluid supply tube 104, and a flow control valve 106. In this example, the cancer-therapy delivery system 100 also includes a saline supply reservoir 108 (or fluid reservoir). The saline can serve as the fluid to be mixed with the microspheres. The saline solution can be at various concentrations such as (0.3%, 0.5%, 0.7%, 0.9%, or the like). In some embodiments, the carrier fluid (typically a saline solution) can also include one or more other components. For example, in some embodiments the carrier fluid can include heparin (in the case of saline, a heparinized saline solution). The cancer-therapy delivery system 100 also includes a fluid injector and withdrawal assembly 110. The cancer-therapy delivery system 100 also includes a radioactive microsphere supply reservoir/mixing chamber 114. In this example, the cancer-therapy delivery system 100 also includes an outflow port 116. The cancer-therapy delivery system 100 also includes a microcatheter 118. FIG. 1 also shows a patient 122 into which the microcatheter 118 can be inserted to deliver the therapeutic suspension of microspheres.

In some embodiments, the microcatheter 118 can specifically be one with a relatively small diameter, such as a microcatheter with a neuro use indication (hereafter “neurocatheter”). In some embodiments, the microcatheter 118 outer diameter can be as small as 0.33 millimeters (mm) (0.013 inches) or even less. However, in other embodiments the catheter or microcatheter can be larger in diameter. In some embodiments, the catheter or microcatheter can have a diameter of less than or equal to 5.33, 4.32, 4.01, 3.66, 3.33, 3.00, 2.67, 2.34, 2.01, 1,68, 1.35, 0.99, 0.66, or even 0.33 mm (0.288, 0.21, 0.17, 0.158, 0.144, 0.131, 0.118, 0.105, 0.092, 0.079, 0.066, 0.053, 0.039, 0.026, or even 0.013 inches (equivalent to 1 Fr)), or a diameter falling within a range between any of the foregoing.

While not intending to be bound by theory, inner diameters greater than a certain point can lead to undesirable microsphere dropout. As such, in various embodiments herein, the inner diameter of the microcatheter (or the inner diameter of a fluid passage within the catheter) can be quite small. For example, in some embodiments, the microcatheter 118 inner diameter can be less than or equal to 0.050, 0.045, 0.040, 0.035, 0.030, 0.035, 0.020, or 0.015 inches (1.27, 1.143, 1.016, 0.889, 0.762, 0.508, or 0.381 mm), or a size falling within a range between any of the foregoing.

In use, various operations can be performed to prepare the system 100. For example, operations can be formed such as system priming, air/bubble removal, flushing operations, and the like. Then (omitting some possible operations for ease of explanation) the clinician or other system user can pull back on a plunger or similar mechanism of therapeutic fluid delivery device 102 causing fluid (such as saline) to be withdrawn from the saline supply reservoir 108, through the flow control valve 106 and the fluid supply tube 104, and into the fluid delivery device 102. Then the clinician or other system user can depress the plunger causing fluid to flow from the therapeutic fluid delivery device 102, through the fluid supply tube 104, through the flow control valve 106, and into the fluid injector and withdrawal assembly 110. The fluid injector and withdrawal assembly 110 can be in fluid communication with the mixing chamber 114 and can direct a flow of fluid into the mixing chamber 114 coming from the therapeutic fluid delivery device 102 or pump such as through one of a pair of needles, cannulas, or tubes serving as an inflow conduit. The fluid can become mixed with microspheres in the mixing chamber 114 forming a suspension which can then exit via the fluid injector and withdrawal assembly 110 via another needle, cannula, or tube serving as an outflow conduit and through tubing and out of the outflow port 116 and into the microcatheter 118 and into a desired site of the patient 122. After an initial volume of fluid is passed through to the patient this way, one or more flushes can be performed (e.g., additional amounts of carrier fluid can be run through the system and to the patient to ensure that all or nearly all of the microspheres are delivered to the patient).

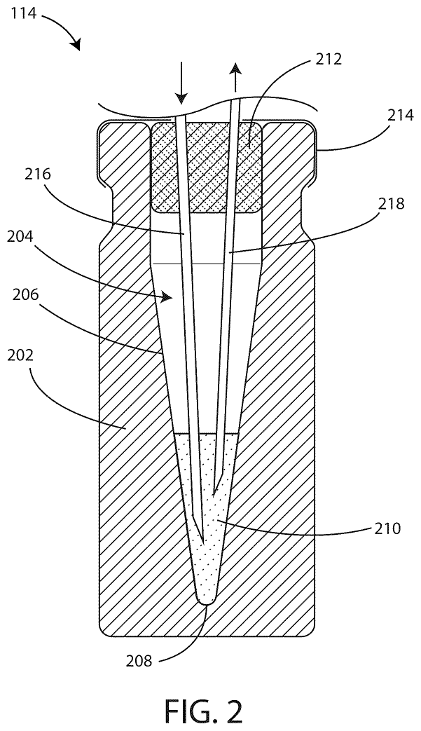

Referring now to FIG. 2, a sectional view is shown of components of a cancer therapy delivery system 100 in accordance with various embodiments herein. The mixing chamber 114 includes a vial 202. The vial 202 can be formed of, for example, a glass, ceramic, or a polymer. In some cases, the vial 202 could even be formed of a metal or a composite. While not shown in this view, in some embodiments the vial 202 can be held within a lead pot to block radiation and a lid for the pot can be removed as part of preparatory operations for the system. The mixing chamber 114 includes an internal volume 204 within the vial 202. The mixing chamber 114 includes internal walls that define the internal volume 204 and those internal walls include a portion with continuously tapered sides 206 (e.g., the taper angle is constant in the area of the continuously tapered sides 206). In horizontal cross-section, the internal volume 204 can be substantially circular. In this example, the internal volume 204 also includes a hemispherical tip 208. As such, the bottom tip of the internal volume 204 is rounded.

The cancer therapy delivery system also includes radioactive microspheres 210 disposed within the vial 202, such as at the bottom of the internal volume 204. The overall volume of the internal volume 204 within the vial 202 can be, in some embodiments, 2 mL, 1.75 mL, 1.5 mL, 1.25 mL, 1 mL, 0.75 mL, 0.6 mL, 0.5 mL, or less, or an amount falling within a range between any of the foregoing. The space within the internal volume 204 above the radioactive microspheres 210 can be filled with a fluid, such as water or saline, in some cases the same fluid as the carrier fluid described above. In some embodiments, the internal volume 204 can contain approximately 1 mL, 0.9 mL, 0.8 mL, 0.7 mL, 0.6 mL, or 0.5 mL, 0.4 mL, 0.3 mL, 0.25 mL, 0.2 mL, or less, of a fluid to start (e.g., within the vial 202 as supplied), or an amount falling within a range between any of the foregoing. The cancer therapy delivery system also includes a septum 212 that serves to seal a top of the vial 202. With the vial 202 sealed by the septum 212 (or similar structure) movement of fluid in through the inflow conduit 216 increases the internal pressure of the vial 202, then forcing fluid out of the outflow conduit 218. The cancer therapy delivery system also includes a retaining cap 214 which serves to retain the septum 212.

The cancer therapy delivery system also includes an inflow conduit 216 and an outflow conduit 218. In use, the inflow conduit 216 and the outflow conduit 218 can start in a first position in which they do not penetrate through the septum 212. For example, they can be withdrawn up into the fluid injector and withdrawal assembly 110 (see FIG. 1). The inflow conduit 216, and the outflow conduit 218 as well, can also be put into a deployed position (manually or automatically) as shown in FIG. 2 where they are inserted through the septum 212 and into the area where the radioactive microspheres 210 are disposed. Thus, in various embodiments, the inflow conduit 216 and/or the outflow conduit 218 can be in selective fluid communication with the mixing chamber 114 and, specifically, the internal volume 204 thereof. The inflow conduit 216 and the outflow conduit 218 can take various forms. For example, in various embodiments, the inflow conduit 216 can be at least one of a needle or a cannula. Similarly, in various embodiments, the outflow conduit 218 can be at least one of a needle or a cannula.

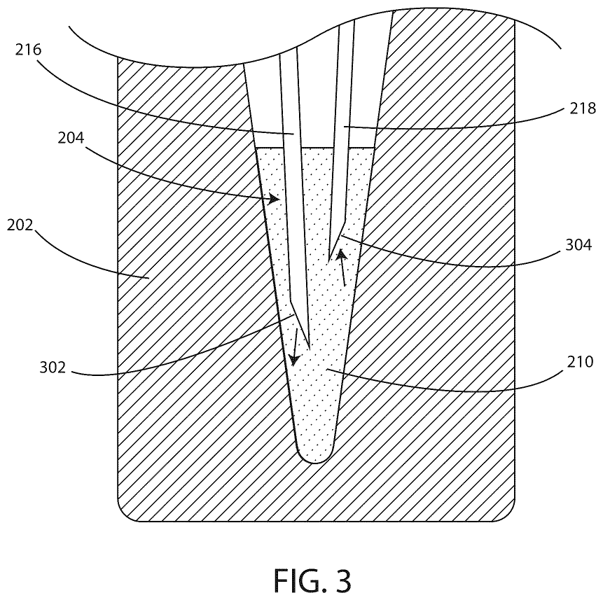

Referring now to FIG. 3, a sectional view of components of a cancer therapy delivery system 100 is shown in accordance with various embodiments herein. Similar to as with FIG. 2, a vial 202 is shown with an internal volume 204. Radioactive microspheres 210 are also shown disposed within the internal volume 204. The cancer therapy delivery system also includes an inflow conduit 216 and an outflow conduit 218. The inflow conduit 216 includes an inflow orifice 302. The inflow orifice 302 can be angled with respect to a lengthwise axis of the inflow conduit 216, or in some embodiments can be substantially perpendicular thereto. Similarly, the outflow conduit 218 includes an outflow orifice 304 that can be angled with respect to a lengthwise axis of the outflow conduit 218 or can be substantially perpendicular thereto. When fluid is pushed through the inflow conduit 216 and into the internal volume 204 (which, with reference to FIG. 2 is sealed by the septum 212 except for the inflow conduit 216 and the outflow conduit 218), such fluid can come out of the outflow orifice 304 (such as in the direction indicated by the arrow in FIG. 3) and mix with the radioactive microspheres 210. Then, as driven by pressure in the internal volume 204, a mixture (or suspension) of the radioactive microspheres 210 and the fluid can be pushed into the outflow orifice 304.

Turbulence and wall shear forces within the vial 202 are driven by the movement of fluid in and out of the vial 202. While not intending to be bound by theory, the generation of relatively high fluid shear conditions at the inner walls of the vial 202 and bulk turbulence in the fluid within the vial 202 has been found to contribute to thorough and consistent mixing and suspension of the radioactive microspheres 210 in the fluid. In turn, it has been found that the geometry of the internal volume contributes to the amount of shear generated, such as on the walls lining the internal volume.

In operation, the vial 202 is generally maintained in a vertical orientation with the vertical axis (or long axis) kept vertical with the hemispherical tip down and the septum 212 up or nearly vertical such as within 10, 7.5, 5, 2.5 or 1 degree of vertical. This leads to the dense microspheres to return to the bottom tip of the vial 202 when the flushing flow of fluid stops. Because of the steep slope inside the vial 202, the microspheres tend to fall back down into the high shear and high turbulence zone near inflow orifice 302 and outflow orifice 304. These aspects are helpful to ensure full clearance of microspheres from the vial 202.

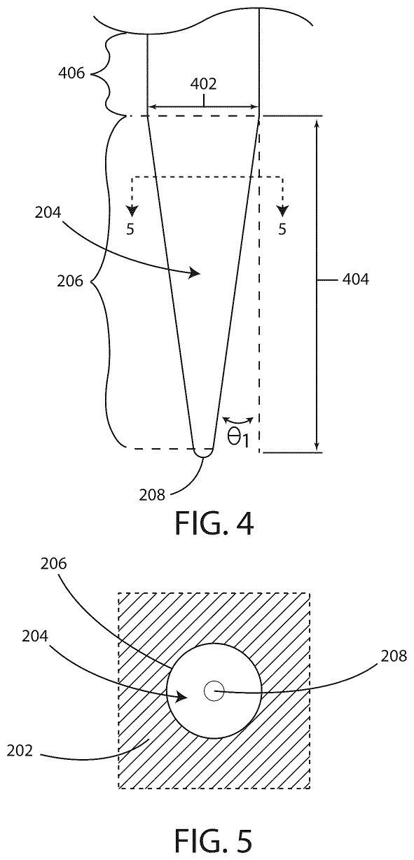

Referring now to FIG. 4, a schematic view of components of a cancer therapy delivery system 100 is shown in accordance with various embodiments herein. The mixing chamber also includes a hemispherical tip 208. The internal volume 204 includes continuously tapered sides 206. The continuously tapered sides 206 include a height 404 and a width 402. While not intending to be bound by theory, it has been found that keeping the taper of the tapered sides 206 relatively steep and keeping the overall shape of the internal volume 204 relatively narrow compared with its height contributes to the generation of high shear conditions. As such, in various embodiments, a width of a portion of the internal volume 204 defining continuously tapered sides 206 is less than 40, 30, 25, or even 20 percent of a height of the portion of the internal volume 204 defining the continuously tapered sides 206, or an amount falling within a range between any of the foregoing. In various embodiments, a region of the internal volume 204 of the mixing chamber with continuously tapered sides 206 has a width to height ratio of less than 1:1, 1:1.5, 1:2, 1:2.5, 1:3, 1:3.5 or even 1:4, or a ratio falling within a range between any of the foregoing.

The continuously tapered sides 206 can be oriented to be relatively steep. This can be quantified by an angle (see θ₁ of FIG. 4) with respect to a lengthwise vertical axis of the mixing chamber. For example, the continuously tapered sides 206 can be at a relatively shallow angle with respect to a lengthwise vertical axis of the mixing chamber 114. More specifically, the continuously tapered sides 206 can be at an angle of less than 20, 15, or even 10 degrees with respect to a lengthwise vertical axis of a mixing chamber 114, or an angle falling within a range between any of the foregoing.

A vertical walled portion 406 can be disposed above the portion with the continuously tapered sides 206. In some embodiments, the point where the vertical walled portion 406 and the portion with the continuously tapered sides 206 meet can be rounded (like a fillet). In some embodiments, the inner surface of the internal volume 204 can lack curved portions other than the hemispherical tip 208 and at the point where the vertical walled portion 406 and the portion with the continuously tapered sides 206 meet.

The overall volume of the internal volume 204 can be relatively small. In some embodiments, an overall volume of the internal volume 204 can be, in some embodiments, 2 mL, 1.75 mL, 1.5 mL, 1.25 mL, 1 mL, 0.75 mL, or less.

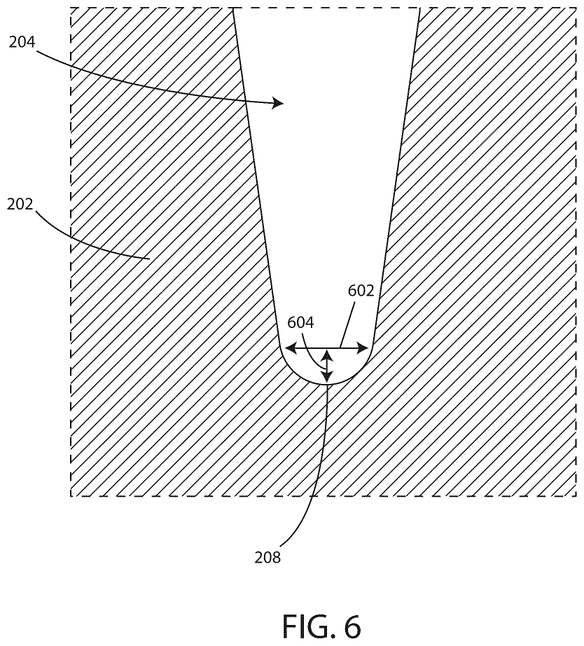

Referring now to FIG. 5, a sectional view is shown of portions of a mixing chamber as taken along line 5-5 of FIG. 4 in accordance with various embodiments herein. In this view, vial 202 is shown having internal volume 204 which is at least partially defined by the tapered sides 206 and the hemispherical tip 208. In this view, it can be seen that the portion of the internal volume 204 in the area of the tapered sides 206 can be substantially circular in cross-section. In some embodiments, the portion of the internal volume 204 in the area of the tapered sides 206 can be perfectly circular in cross-section. However, other shapes are also contemplated herein.

Referring now to FIG. 6, an enlarged view of a portion of the mixing chamber including the hemispherical tip 208 is shown. In this view, the vial 202 is shown along with the internal volume 204. The hemispherical tip 208 is disposed at the bottom of the internal volume 204. In various embodiments, the hemispherical tip 208 can be substantially hemispherical. In this example, the width 602 (or diameter) of the top of the hemispherical tip 208 portion is twice the depth 604 resulting in a perfect hemisphere. It will be appreciated, however, that in some embodiments, the hemispherical tip 208 can take on other proportions. For example, in some embodiments, the width 602 can be more than twice the depth 604 resulting in a shallower hemispherical shape. In some embodiments, the width 602 can be less than twice the depth 604 resulting in a deeper hemispherical shape.

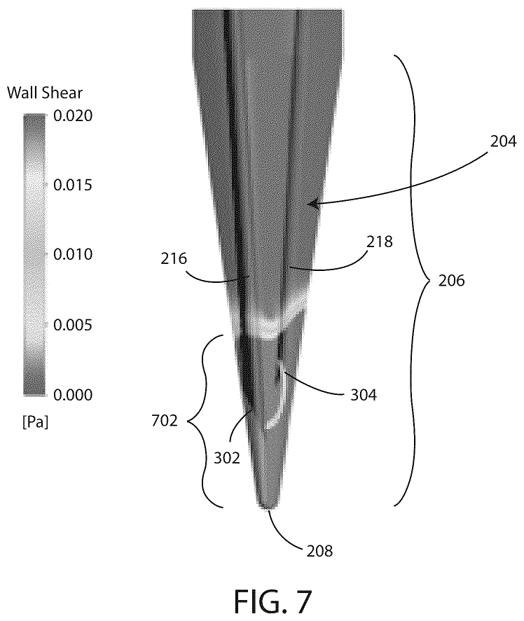

Referring now to FIG. 7, a schematic view is shown of portions of a mixing chamber 114 illustrating the amounts of shear generated at surfaces in accordance with various embodiments herein. As before, the internal volume includes continuously tapered sides 206. Also, the cancer therapy delivery system is shown with inflow conduit 216, inflow orifice 302, an outflow conduit 218, and outflow orifice 304.

The continuously tapered sides 206 includes a high shear region 702 wherein wall shear values are 0.020 Pa or greater. As such, in various embodiments herein, the fluid that flows into the fluid reservoir 108 generates a region of high shear along the walls thereof of at least 0.020 Pa when a fluid flows through the fluid reservoir 108 at a rate of approximately 5 to 7 mL/minute, or about 6 mL/minute. The region of high shear extends from the bottom of the hemispherical tip 208 to a point above the bottom of the outflow conduit 218 when the outflow conduit 218 is in a lowered, deployed position.

### Microspheres

Microspheres herein can include those with a combination of yttria, alumina, and silica. By way of example, in some embodiments, microspheres herein can include Y₂O₃-Al₂O₃—SiO₂ in a 40:20:40 wt. % ratio. As such, in various embodiments, the microspheres herein can be glass microspheres. It will be appreciated however, that other types of microspheres are also contemplated herein.

In some embodiments, microspheres can be prepared by combining yittrium-89 with alumina and silica, in some cases also using a flame spheroidization method, and using neutron bombardment to convert Y-89 into the beta emitting radioisotope Y-90, making the microspheres radioactive microspheres. In various embodiments, the amount of beta radiation can exceed 2500, 3000, 4000, 5000, 6000, 7000, 8000, or even 9000 Bq per sphere at the time of activity calibration (recognizing that the amount of radiation will drop after that point as the Y-90 radioisotope decays). In some embodiments, the microspheres can be provided in a vial with activity of 3 GBq or lower up to 20 GBq or higher (at calibration time or “reference date and time”). However, in some embodiments, the microspheres can be provided in a vial with activity of less than 3, 2.75, 2.5, 2.25, 2, 1.75, 1.5, 1.25, 1.0, 0.75, 0.5, 0.4, 0.3, 0.35, 0.2, 0.15, or 0.1 GBq, or less at calibration time, or an amount falling within a range between any of the foregoing.

It will be appreciated that dosages can vary based on factors including the type of tumor/tissue to be treated, location of the tumor/tissue to be treated, factors specific to a particular patient, and the like. In some embodiments the dosage of the therapy can be less than or equal to 5000 Gy, 4500 Gy, 4000 Gy, 3500 Gy, 3000 Gy, 2500 Gy, 2000 Gy, 1500 Gy, 100 Gy, 500 Gy, 400 Gy, 300 Gy, 250 Gy, 225 Gy, 200 Gy, 180 Gy, 150 Gy, 120 Gy, 100 Gy, 90 Gy, 80 Gy, 70 Gy, 60 Gy, 50 Gy, 40 Gy, 30 Gy, or 20 Gy, or an amount falling within a range between any of the foregoing.

The size of the microspheres can be extremely small. In some embodiments, the average diameter of the microspheres can be from about 20 micrometers (μm) to about 30 μm. However, in some embodiments the microspheres can be somewhat smaller or larger.

The density of the microspheres (such as glass microspheres) can be quite high. In some embodiments, the density of the microspheres can be above 3 g/mL, such as from 3.1 to 3.5 g/mL, or about 3.3 g/mL. By comparison, the density of water at room temperature is about 0.9978 g/mL. As such, the density of microspheres is much higher than a saline solution which influences how readily such microspheres can settle out of a suspension.

### Methods

Many different methods are contemplated herein, including, but not limited to, methods of making, methods of using, methods of administering a cancer therapy, methods of mixing a cancer therapy microsphere with a carrier fluid and the like. Aspects of system/device operation described elsewhere herein can be performed as operations of one or more methods in accordance with various embodiments herein.

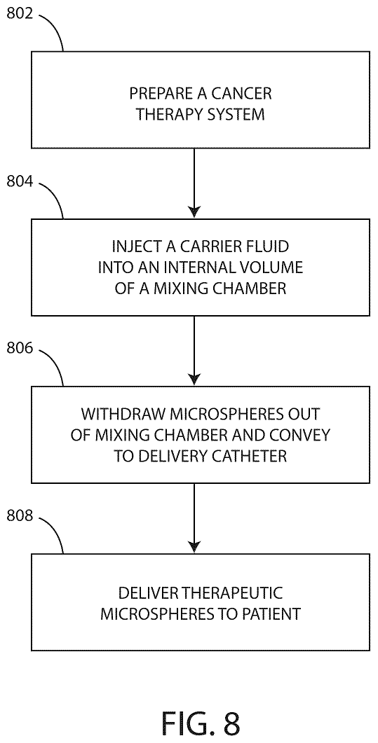

Referring now to FIG. 8, in some embodiments a method of administering a cancer therapy can include preparing a cancer-therapy delivery system 802 (including, for example, one or more operations such as priming the system, removing bubbles, flushing the lines, and the like). In addition, the method can include injecting or otherwise flowing a carrier fluid into an internal volume of a mixing chamber containing therapeutic radioactive microspheres 804. The method can also include, withdrawing a mixture or suspension of therapeutic radioactive microspheres and carrier fluid out of the internal volume of the mixing chamber and conveying the same to a delivery catheter 806. The method can also include delivering therapeutic radioactive microspheres to a patient using via the delivery catheter 808.

In an embodiment of the method, cancer therapy is delivered to treat a brain tumor. In an embodiment of the method, cancer therapy is delivered to treat glioblastoma. However, treatment of other types of tumors is also contemplated herein. By way of example, tumors and/or tissue of the head or neck, liver, breast, cervix, prostate, or eye, as well as other tissues can be treated in accordance with embodiments herein. In some embodiments it can be used to treat non-cancerous tumors or other tissue.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A cancer therapy delivery system comprising:
a mixing chamber, the mixing chamber;
defining a hemispherical tip; and
defining continuously tapered sides;
an inflow conduit, wherein the inflow conduit is in selective fluid communication with the mixing chamber; and
an outflow conduit, wherein the outflow conduit is in selective fluid communication with the mixing chamber.

2. The cancer therapy delivery system of claim 1, wherein a width of a portion of the mixing chamber defining the continuously tapered sides is less than 30 percent of a height of the portion of the continuously tapered sides.

3. The cancer therapy delivery system of claim 1, further comprising:
a pump; and
a fluid reservoir, wherein the fluid is withdrawn from the fluid reservoir and flows into the mixing chamber via the inflow conduit.

4. The cancer therapy delivery system of claim 3, wherein the fluid is a saline solution.

5. The cancer therapy delivery system of claim 3, further comprising radioactive microspheres, wherein the radioactive microspheres are disposed within the mixing chamber.

6. The cancer therapy delivery system of claim 5, wherein the radioactive microspheres have a density of about 3.3 g/mL.

7. The cancer therapy delivery system of claim 5, wherein the radioactive microspheres have an average diameter of about 20 μm to about 30 μm.

8. The cancer therapy delivery system of claim 5, wherein the fluid that flows into the fluid reservoir causes the radioactive microspheres to form a suspension with the glass microspheres that then passes out of the mixing chamber via the outflow conduit.

9. The cancer therapy delivery system of claim 3, wherein the fluid that flows into the fluid reservoir generates a region of shear along the walls thereof of at least 0.020 Pa when the fluid flows through the fluid reservoir at a rate of at least 5 mL/minute.

10. The cancer therapy delivery system of claim 3, wherein the fluid that flows into the fluid reservoir generates a region of shear along the walls thereof of at least 0.020 Pa covering a region extending above the bottom of the outflow conduit when the outflow conduit is in a deployed position when the fluid flows through the fluid reservoir at a rate of at least 5 mL/minute.

11. The cancer therapy delivery system of claim 1, wherein the continuously tapered sides are at an angle of less than 20 degrees with respect to a lengthwise vertical axis of the mixing chamber.

12. The cancer therapy delivery system of claim 1, wherein the continuously tapered sides are at an angle of less than 10 degrees with respect to a lengthwise vertical axis of the mixing chamber.

13. The cancer therapy delivery system of claim 1, wherein a region of the mixing chamber having the continuously tapered sides has a width to height ratio of less than 1:2.

14. The cancer therapy delivery system of claim 1, wherein a region of the mixing chamber having the continuously tapered sides has a width to height ratio of less than 1:3.

15. The cancer therapy delivery system of claim 1, wherein a volume of the mixing chamber up to a top of the continuously tapered sides is less than 1 mL.

16. The cancer therapy delivery system of claim 1, wherein the mixing chamber is defined by a glass or polymeric vial.

17. The cancer therapy delivery system of claim 1, wherein the inflow conduit is at least one of a needle or a cannula.

18. The cancer therapy delivery system of claim 1, wherein the outflow conduit is at least one of a needle or a cannula.

19. A method of delivering a cancer therapy to a patient comprising:
preparing a cancer therapy system;
injecting a carrier fluid into an internal volume of a mixing chamber of the cancer therapy system, wherein the mixing chamber defines a hemispherical tip and continuously tapered sides;
withdrawing microspheres out of the mixing chamber in the form of a mixture or suspension with the carrier fluid and conveying the same to a delivery catheter; and
delivering the therapeutic microspheres to the patient using the delivery catheter.

20. The method of delivering a cancer therapy to a patient of claim 19, wherein preparing a cancer therapy system includes one or more of one or more operations including priming the system, removing bubbles, and flushing the lines.
